(19) United States
(12) Patent Application Publication
(10) Pub. No.: US 2005/0187437 A1
(43) Pub. Date: Aug. 25, 2005

(54) Information processing apparatus and method

(76) Inventors: Masakazu Matsugu, Chiba-shi (JP); Katsuhiko Mori, Kawasaki-shi (JP); Yuji Kaneda, Kitakyushu-shi (JP)

Correspondence Address: Cowan, Liebowitz & Latman, P.C., 1133 Avenue of the Americas, New York, NY, 10036-6799, US

(21) Appl. No.: 11/064,624

(22) Filed: Feb. 24, 2005

(30) Foreign Application Priority Data
Feb. 25, 2004 (JP) 2004-049934

(51) Int. Cl. A61B 5/00; A61B 5/04; A61B 10/00

(52) U.S. Cl. 600/301; 128/920; 128/905

(57) ABSTRACT

An information processing apparatus detects the facial expression and body action of a person image included in image information, and determines the physical/mental condition of the user on the basis of the detection results. Presentation of information by a presentation unit which visually and/or audibly presenting information is controlled by the determined physical/mental condition of the user.

### FIELD OF THE INVENTION

The present invention relates to an information service using a multimodal interface which is controlled by detecting the physical/mental conditions of a person such as facial expressions, actions, and the like, which are expressed non-verbally and tacitly.

### BACKGROUND OF THE INVENTION

A system (see Japanese Patent Laid-Open No. 2002-334339) which activates sensitivity of the user by controlling presentation of stimuli on the basis of the history of changes in condition (facial expression, line of sight, body action, and the like) of the user, a biofeedback apparatus (see Japanese Patent Laid-Open No. 2001-252265) and biofeedback game apparatus (see Japanese Patent Laid-Open No. 10-328412) which change the mental condition of a player, and the like have been proposed. Japanese Patent Laid-Open No. 10-71137 proposes an arrangement which detects the stress level on the basis of fluctuation of heart rate intervals obtained from a pulse wave signal, and aborts the operation of an external apparatus such as a computer, game, or the like when the rate of increase in stress level exceeds a predetermined value. A multimodal interface apparatus disclosed in Japanese Patent Laid-Open No. 11-249773 controls interface operations by utilizing nonverbal messages to attain natural interactions.

Of the aforementioned techniques, the multimodal interface apparatus is designed in consideration of how to effectively and precisely use gestures and facial expressions for operations and instructions, intentionally given by the user. However, the multimodal interface apparatus does not have as its object to provide an interface function that provides a desired or predetermined information service by detecting the intention or condition non-verbally and tacitly expressed by the user.

The sensitivity activation system effectively presents effective stimuli for, e.g., rehabilitation on the basis of the history of feedback of the user to simple stimuli, but cannot provide an appropriate information service in correspondence with the physical/mental conditions of the user. The stress detection method used in, e.g., a biofeedback game or the like detects only biofeedback of a player, but cannot precisely estimate various physical/mental condition levels other than stress. As a result, it is difficult for this method to effectively prevent physical/mental problems such as wandering attention after the game, epileptic fit or the like, and so forth. Since the sensitivity activation system, biofeedback game, and the like use only biological information, they can detect specific physical/mental conditions (e.g., stress, fatigue level, and the like) of the user but can hardly detect a large variety of physical/mental conditions.

### SUMMARY OF THE INVENTION

The present invention has been made in consideration of the aforementioned problems, and has as its object to allow to use information associated with facial expressions and actions acquired from image information, and to precisely detect tacit physical/mental conditions.

It is another object of the present invention to control presentation of information corresponding to user's conditions by precisely detecting tacit physical/mental conditions using speech and/or biological information and the like together with the information associated with facial expressions and actions in a comprehensive manner.

According to one aspect of the present invention, there is provided an information processing apparatus comprising: a first detection unit configured to detect a facial expression and/or body action of a user included in image information; a determination unit configured to determine a physical/mental condition of a user on the basis of the detection result of the first detection unit; a presentation unit configured to visually and/or audibly present information; and a control unit configured to control presentation of the information by the presentation unit on the basis of the physical/mental condition of the user determined by the determination unit.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

The accompanying drawings, which are incorporated in and constitute a part of the specification, illustrate embodiments of the invention and, together with the description, serve to explain the principles of the invention.

FIG. 1 is a block diagram showing the arrangement of an information presentation apparatus according to the first embodiment;

FIG. 2 is a flowchart for explaining the principal sequence of an information presentation process according to the first embodiment;

FIG. 3 is a block diagram showing the arrangement of an image recognition unit 15;

FIG. 4 is a block diagram showing the arrangement of a biological information sensing unit 12;

FIG. 5 is a flowchart for explaining the information presentation process according to the first embodiment;

FIG. 6 is a block diagram showing the arrangement of an information presentation system according to the second embodiment;

FIG. 7 is a flowchart for explaining the information presentation process according to the second embodiment; and

FIGS. 8A and 8B illustrate the configurations of contents according to the fourth embodiment.

### DETAILED DESCRIPTION OF THE PREFERRED EMBODIMENTS

Preferred embodiments of the present invention will now be described in detail in accordance with the accompanying drawings.

### First Embodiment

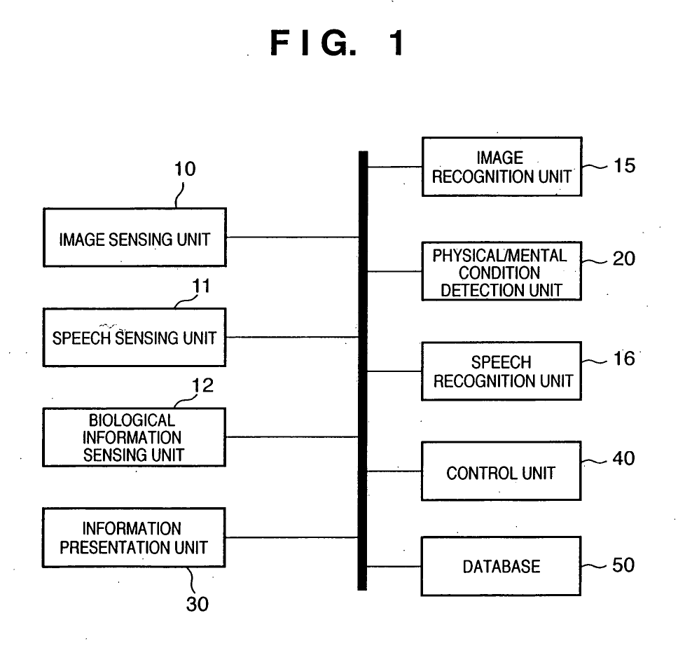

The first embodiment of the present invention will be described in detail hereinafter with reference to the accompanying drawings. FIG. 1 is a block diagram showing the arrangement of principal part of an information presentation system according to the first embodiment. The information presentation system comprises an image sensing unit 10 (including an imaging optical system, video sensor, sensor signal processing circuit, and sensor drive circuit), speech sensing unit 11, biological information sensing unit 12, image recognition unit 15, speech recognition unit 16, physical/mental condition detection unit 20, information presentation unit 30, control unit 40 which controls these units, database unit 50, and the like. With the above arrangement, in this embodiment, the user's physical/mental conditions are roughly estimated on the basis of image information obtained from the image recognition unit 15, and the physical/mental conditions are estimated in detail using the estimation result, speech information, biological information, and the like. An overview of the functions of the respective units will be explained below.

The image sensing unit 10 includes an image sensor that senses a facial image of a person or the like as a principal component. The image sensor typically uses a CCD, CMOS image sensor, or the like, and outputs a video signal in response to a read control signal from a sensor drive circuit (not shown). The speech sensing unit 11 comprises a microphone, and a signal processing circuit for separating and extracting a user's speech signal input via the microphone from a background audio signal. The speech signal obtained by the speech sensing unit 11 undergoes speech recognition by the speech recognition unit 16, and its signal frequency or the like is measured by the physical/medical condition detection unit 20.

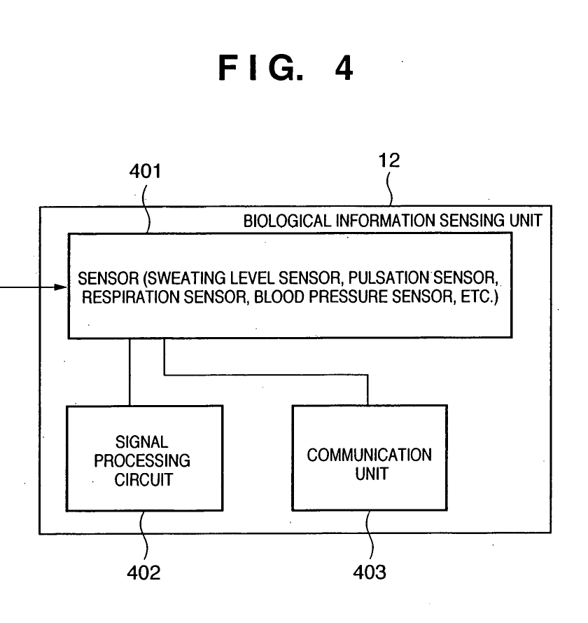

The biological information sensing unit 12 comprises a sensor 401 (including at least some of a sweating level sensor, pulsation sensor, expiratory sensor, respiration pattern detection unit, blood pressure sensor, iris image input unit, and the like) for acquiring various kinds of biological information, a signal processing circuit 402 for generating biological information data by converting sensing data from the sensor 401 into an electrical signal and applying predetermined pre-processes (compression, feature extraction, and the like), and a communication unit 403 (or data line) for transmitting the biological information data obtained by the signal processing circuit 402 to the information presentation unit 30 and control unit 40, as shown in FIG. 4. The estimation precision of the physical/medical conditions to be described later can be improved by sensing and integrating a variety of biological information. Note that this biological information sensing unit 12 may be worn by a human body or may be incorporated in this information presentation system. When this unit 12 is worn by a human body, it may be embedded in, e.g., a wristwatch, eyeglasses, hairpiece, underwear, or the like.

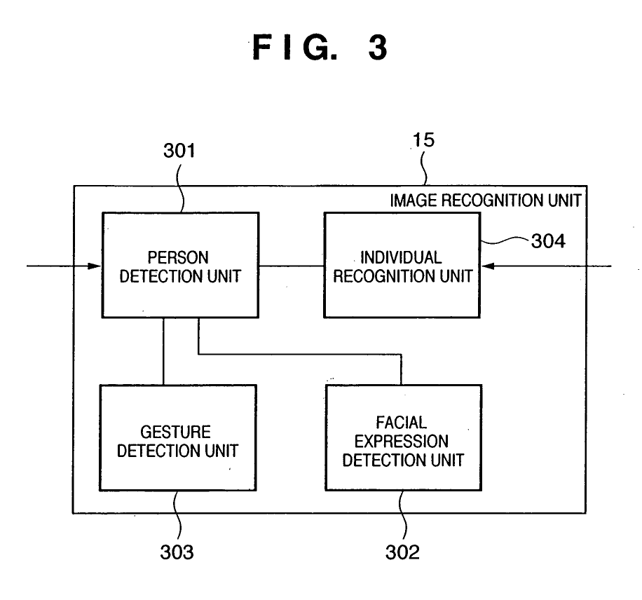

The image recognition unit 15 has a person detection unit 301, facial expression detection unit 302, gesture detection unit 303, and individual recognition unit 304, as shown in FIG. 3. The person detection unit 301 is an image processing module (software module or circuit module) which detects the head, face, upper body, or whole body of a person by processing image data input from the image sensing unit 10. The individual recognition unit 304 is an image processing module which specifies a person (registered person) (to identify the user) using the face or the like detected by the person detection unit 301. Note that algorithms of head/face detection, face recognition (user identification), and the like in these image processing modules may adopt known methods (e.g., see Japanese Patent No. 3078166 by the present applicant).

The facial expression detection unit 302 is an image processing module which detects predetermined facial expressions (smile, bored expression, excited expression, perplexed expression, angry expression, shocked expression, and the like). The gesture detection unit 303 is an image processing module which detects specific actions (walk, sit down, dine, carry a thing, drive, lay down, fall down, pick up the receiver, grab a thing, release, and the like), changes in posture, specific hand signals (point, beck, paper-rock-scissors actions, and the like), and so forth. As for the facial expression recognition technique and gesture detection technique, known methods can be used.

Referring back to FIG. 1, the physical/mental condition detection unit 20 performs first estimation of the physical/mental conditions using the recognition result of the image recognition unit 15. This first estimation specifies candidates of classifications of conditions (condition classes) of a plurality of potential physical/mental conditions. Furthermore, the physical/mental condition detection unit 20 narrows down the condition classes of the physical/mental conditions obtained as the first estimation result using output signals from various sensing units (speech sensing unit 11 and/or biological information sensing unit 12) to determine the condition class of the physical/mental condition of the user and also determine a level in that condition class (condition level). In this way, the physical/mental conditions are roughly estimated on the basis of image information which appears as apparent conditions, and the conditions are narrowed down on the basis of the biological information and speech information extracted by the speech sensing unit 11/biological information sensing unit 12, thus estimating the physical/mental condition (determining the condition class and level). Hence, the estimation precision and processing efficiency of the physical/mental condition detection unit 20 can improve compared to a case wherein its process is done based on only sensing data of biological information. Note that the first estimation may determine one condition class of the physical/mental condition, and second estimation may determine its condition level.

In this specification, the physical/mental conditions are state variables which are expressed as facial expression and body actions of the user in correspondence with the predetermined emotions such as delight, anger, sorrow, and pleasure, or the interest level, satisfaction level, excitation level, and the like, and which can be physically measured by the sensing units. For example, when the interest level and excitation level increase, numerical values such as a pulse rate, sweating level, pupil diameter, and the like rise. When the satisfaction level increases, a facial expression such as smile or the like and a body action such as nod or the like appear. When a person is good humored, the center frequency level of speech increases, and state changes such as eyes slanting down, smiling, and the like are observed. When a person is irritated, actions such as shaking oneself nervously, tearing one's hair, and the like are observed by the image recognition unit 15.

Note that the pulse rate, blood pressure, sweating amount, and speech have individual differences. Hence, these data in a calm state are stored in the database unit, and upon detection of changes in physical/mental conditions, evaluation values associated with deviations are calculated from these reference data. Hence, the physical/mental conditions are estimated based on these deviations. That is, data in a calm state are stored individually, and evaluation values are calculated using the data in a calm state corresponding to an individual specified by the individual recognition unit 304.

Also, the physical/mental condition detection unit 20 includes processing modules (excitation level estimation module, happiness level estimation module, fatigue level estimation module, satisfaction level estimation module, interest level estimation module) and the like that estimate not only the types of physical/mental conditions but also their levels (excitation level, satisfaction level, interest level, fatigue level, and the like) on the basis of various kinds of sensing information. For example, the “excitation level” is estimated by integrating at least one or a plurality of the heart rate and respiration frequency level (or irregularity of pulse wave and respiration rhythm), facial expressions/actions such as blushing, laughing hard, roaring, and the like, and sensing information of speech levels such as a laughing voice, roar of anger, cry, gasping, and the like, as described above. The “interest level” can be estimated by the size of the pupil diameter, an action such as hanging out or the like, the frequency and time width of gazing, and the like. The “satisfaction level” can be estimated by detecting the magnitude of nod, words that express satisfaction or feeling of pleasure (“delicious”, “interesting”, “excellent”, and the like) and their tone volumes, or specific facial expressions such as smile, laughing, and the like.

The physical/mental conditions may be estimated using only processing information (detection information associated with a facial expression and gesture obtained from the image recognition unit 15) from the image sensing unit 10. In general, however, the physical/mental conditions are estimated and categorized by integrating a plurality of pieces of processing information (e.g., the heart rate, facial expression, speech, and the like) from a plurality of other sensing units. As that processing method, known techniques such as a neural network (a self assembly map, support vector machine, radial basis function network, the other feedforward or recurrent type parallel hierarchical processing models, and the like), statistical pattern recognition, a statistical method such as multivariate analysis or the like, a technique such as so-called sensor fusion or the like, Bayesian Network, and so forth can be used.

The information presentation unit 30 incorporates a display and loudspeaker (neither are shown), a first storage unit (not shown) for storing information presentation programs, and a second storage unit (not shown) for storing user's preference. Note that the information stored in these storage units may be stored in the database unit 50.

The control unit 40 selectively launches the information presentation program set in advance in the information presentation unit 30 in correspondence with the estimated physical/mental condition based on the output from the physical/mental condition detection unit 20, stops or aborts current information presentation, displays information corresponding to the estimated condition of the user, and so forth. Information presentation is stopped or aborted when a dangerous state or forerunner (maximum fatigue, indication of cardiac failure, or the like) of the physical/mental condition is automatically detected and avoided.

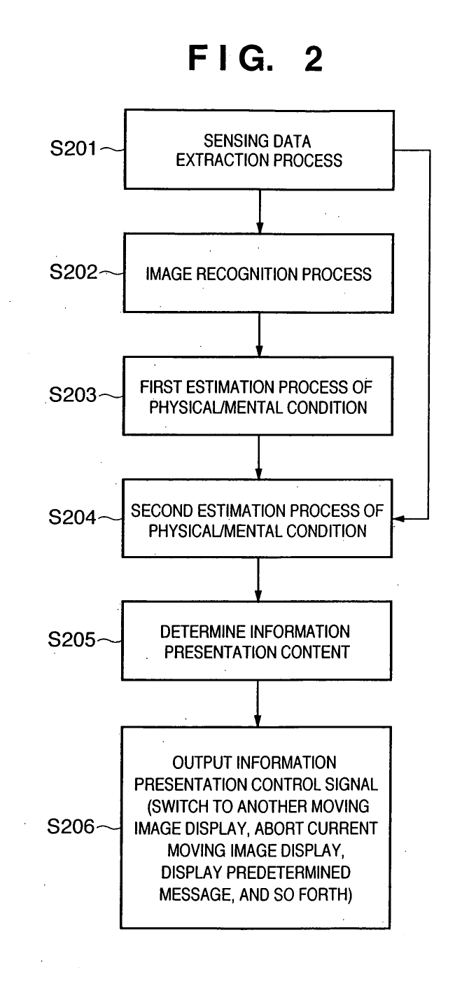

FIG. 2 is a flowchart that summarizes the basic processing flow in the first embodiment. An extraction process for acquiring sensing data (image, speech, and biological information data) from the image sensing unit 10, speech sensing unit 12, and biological information sensing unit 13 is executed (step S201). The image recognition unit 15 executes image recognition processes such as person detection, individual recognition, facial expression recognition, action recognition, and the like (step S202). The physical/mental condition detection unit 20 executes a first estimation process of physical/mental conditions on the basis of the image recognition result of the image recognition unit 15 (step S203). The physical/mental condition detection unit 20 also performs second estimation on the basis of the first estimation result in step S203, and sensing information other than the facial expression recognition and action recognition (i.e. sensing information other than image data such as speech and biological information, information obtained from an iris image and the like) (step S204). The information presentation content is determined (including a change in presentation content, and start and stop of information presentation) on the basis of the type (condition class) of the physical/mental condition and its degree (condition level) obtained by this second estimation (step S205), thus generating an information presentation control signal (step S206).

In this embodiment, information presentation indicates services of contents such as music, movies, games, and the like. For example, when yawning as a body action of the user, a hollow, bored expression, and the like are observed by the image recognition unit 15, the physical/mental condition detection unit 20 outputs the first estimation result indicating a high boredom level (condition class=boredom). Furthermore, the second estimation estimates the level of boredom using a yawning voice detected by the speech sensing unit 11 and the calculation result of an awakening level, which is estimated by calculating a pupillogram obtained from the pupil diameter by the biological information sensing unit 12. The control unit 40 switches to contents of another genre and visually or audibly outputs a message that asks a question about the need to abort information presentation or the like on the basis of this estimation result (the condition level of boredom in this case).

In this way, the control unit 40 controls the content of information to be presented by the information presentation unit 30 on the basis of the output (second estimation result) from the physical/mental condition detection unit 20. More specifically, the control unit 40 generates a control signal (to display a message that prompts the user to launch, stop, or abort, and so forth) associated with presentation of an image program prepared in advance in correspondence with the first condition class (bored condition, excited condition, fatigue condition, troubled condition, or the like) as the estimated class of the physical/mental condition, which is obtained as a result of the first estimation of the physical/mental condition detection unit 20 on the basis of the output from the image recognition unit 15, and the second condition class as the estimated class of the physical/mental condition, which is obtained as a result of second estimation using the output from the speech sensing unit 11 or biological information sensing unit 12, and its level (boredom level, excitation level, fatigue level, trouble level, or the like). The contents of control signals corresponding to the condition classes and levels of the physical/mental conditions are stored as a lookup table in the database unit 50 or a predetermined memory (not shown). Upon detection of the fatigue level, malaise, fear, or disgust of a predetermined level or higher, the control unit 40 switches to display of another moving image, stops display of the current moving image, or displays a predetermined message (alert message “the brain fatigues. Any more continuation will harm your health” or the like). That is, the information presentation unit 30 presents information detected in association with the physical/mental condition of the user.

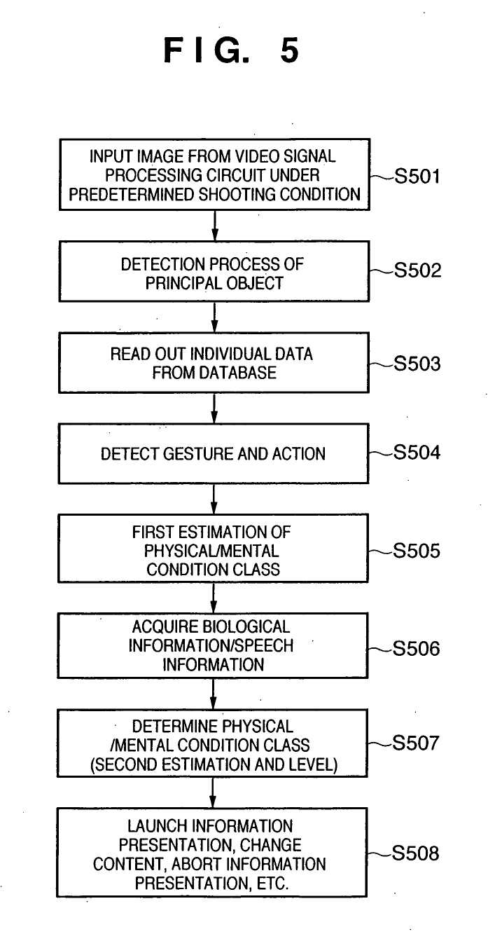

The physical/mental condition detection process according to the first embodiment will be described in more detail below with reference to the flowchart of FIG. 5.

In step S501, the image recognition unit 15 receives an image from the image sensing unit 10. In step S502, the person detection unit 301 detects a principal object (person's face) from the input image. In step S503, the individual recognition unit 304 specifies the detected person, i.e., performs individual recognition, and individual data of biological information (heart rhythm, respiration rhythm, blood pressure, body temperature, sweating amount, and the like), speech information (tone of voice or the like), and image information (facial expressions, gestures, and the like) corresponding to respective physical/mental conditions associated with that person are loaded from the database unit 50 and the like onto a primary storage unit on the basis of the individual recognition result.

Note that primary feature amounts extracted for a pre-process of the person detection and recognition processes in steps S502 and S503 include feature amounts acquired from color information and motion vector information, but the present invention is not limited to those specific feature amounts. Other feature amounts of lower orders (for example, geometric features having a direction component and spatial frequency of a specific range, or local feature elements or the like disclosed in Japanese Patent No. 3078166 by the present applicant) may be used. Note that the image recognition process may use, e.g. a hierarchical neural network circuit (Japanese Patent Laid-Open Nos. 2002-008032, 2002-008033, and 2002-008031) by the present applicant, and other arrangements. When no user is detected within a frame, non-detection signal of a principal object may be output.

If no individual can be specified in step S503, lookup table data prepared in advance as general-purpose model data are loaded.

In step S504, the image recognition unit 15 detects a predetermined facial expression, gesture, and action from the image data input using the image sensing unit 10 in association with that person. In step S505, the physical/mental condition detection unit 20 estimates the condition class of the physical/mental condition (first estimation) on the basis of the detection results of the facial expression, gesture, and action output from the image recognition unit 15 in step S504. The physical/mental condition detection unit 20 then acquires signals from the speech sensing unit 11 and biological information sensing unit 12 in step S506, and performs second estimation on the basis of the first estimation result and these signals in step S507. That is, the condition classes obtained by the first estimation are narrowed down, and the class and level of the physical/mental condition are finally determined. In step S508, the control unit 40 aborts or launches information presentation, displays an alert message or the like, changes the information presentation content, changes the story development speed of the information presentation content, changes the difficulty level of the information presentation content, changes the text size for information presentation, and so forth on the basis of the determined physical/mental condition class and level (condition level).

For example, the change in difficulty level of the information presentation contents means a change to hiragana or plain expression when the estimation result of the physical/mental condition is the “trouble” state and its level value exceeds a predetermined value. Likewise, the text size for information presentation is changed when a facial expression such as narrowing the eyes or the like or an action such as moving the face toward the screen or the like is detected (to change the text size to be displayed to increase). Upon launching information presentation, when the estimated physical/mental condition is “boredom”, “depression”, or the like and its level value exceeds a predetermined value, an information presentation program (movie, game, music, education, or the like) that allows the user to break away from that physical/mental condition and activates his or her mental act is launched. The information presentation program may be interactive contents (interactive movie, game, or education program). The information presentation is aborted when the detected physical/mental condition is “fatigue” or the like with a high level, i.e., the user faces with the physical/mental condition which is set in advance that any more continuation is harmful.

Such information presentation control may be made to maintain the user's physical/mental condition within a predetermined activity level range estimated from the biological information, facial expression, and the like.

As described above, according to the first embodiment, the physical/mental conditions are recognized (first estimation) on the basis of the facial expression and body action expressed by the user, and the physical/mental conditions are narrowed down on the basis of sensing information other than the facial expression and body action (speech information and sensing information, image information such as an iris pattern or the like) to determine the condition class and level of the physical/mental condition (second estimation). Hence, the physical/mental condition can be efficiently and precisely determined. Since information presentation to the user is controlled on the basis of the condition class and level of the physical/mental condition determined in this way, appropriate information corresponding to the user's physical/mental condition can be automatically presented.

### Second Embodiment

In the first embodiment, presentation of information stored in the database unit 50 of the apparatus is controlled in accordance with the physical/mental condition detected by the physical/mental condition detection unit 20. In the second embodiment, a case will be examined wherein information to be presented is acquired from an external apparatus.

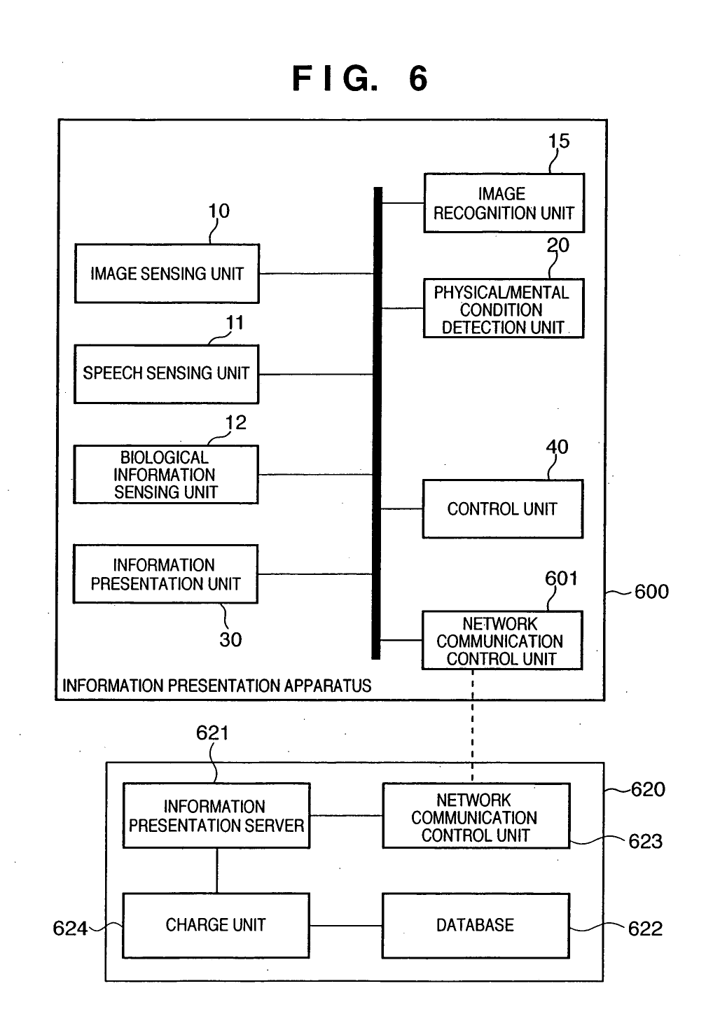

FIG. 6 is a block diagram showing the arrangement of an information presentation system according to the second embodiment. In FIG. 6, the same reference numerals denote the same components as those in the arrangement of the first embodiment (FIG. 1). In the second embodiment, a network communication control unit 601 that makes communication with the network is provided in place of the database unit 50. The information presentation unit 30 accesses an external apparatus 620 via the network communication control unit 601 using the condition level of the physical/mental condition detected by the physical/mental condition detection unit 20 as a trigger, and acquires information to be presented in correspondence with that condition level. Note that the speech recognition unit 16 may be provided as in FIG. 1.

In the external apparatus 620, a network communication control unit 623 can communicate with an information presentation apparatus 600 via the network. An information presentation server 621 acquires corresponding information from a database 622 on the basis of an information request received from the information presentation apparatus 600, and transmits it to the information presentation apparatus 600. A charge unit 624 charges for information presentation. Note that the information presentation unit 30 may specify required information in accordance with the condition level of the physical/mental condition, and may request the external apparatus 620 to send it, or the unit 30 may transmit the detected condition level of the physical/mental condition together with an information request, and the information presentation server 621 of the external apparatus 620 may specify information according to the received physical/mental condition.

An application example of the second embodiment will be explained. This application example will explain a system and service that perform image conversion according to a predetermined facial expression and body action, and providing the image by using the information presentation unit 30. An interface function that automatically performs image conversion which is triggered by a predetermined bodily change of the user, is implemented.

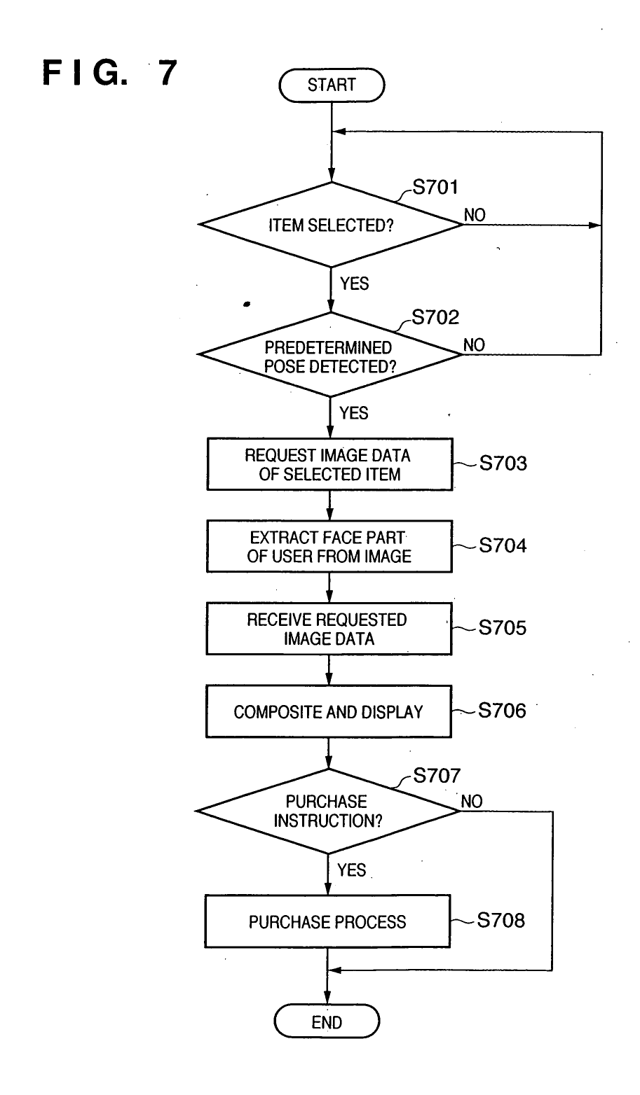

This system implements a sales system via the Internet. FIG. 7 is a flowchart for explaining the process according to the second embodiment. When the user who wants to purchase a wear, headwear, eyeglasses, or the like browses a brochure on the window via the Internet, selects an item to his or her liking, and makes a predetermined facial expression or pose, the flow advances to step S703 via steps S701 and S702. In step S703, a request of image data associated with the selected item is issued to the external apparatus 620. In step S704, the head or whole body image of that user is extracted, and the extracted image is held by the information presentation apparatus 600 (the extracted image and full image may be held). On the other hand, since the information presentation server 621 on the center side transmits display data of the item selected from the brochure to the user terminal via the communication line, the display data is received in step S705, and is displayed on the information presentation unit 30 (display) of the information presentation apparatus 600. A composite image generation program is installed in the information presentation unit 30, and composites the item image received in step S705 to the image of the user who makes the predetermined facial expression or pose extracted in step S704 to generate an image of the user who wears that item, thus displaying the generated image on the information presentation unit 30 (display) (step S706). When the user confirms that image and finally instructs to determine the purchase, the flow advances from step S707 to step S708 to achieve the purchase of the item. Note that the charge unit 624 is used for the purpose of charging for a service that provides various composite image data as well as charging upon purchasing an item by the user.

In the above description, information of the facial expression and body action is used as a trigger for acquiring image data from the external apparatus. Alternatively, whether or not such information is used as a trigger may be determined in consideration of other kinds of information, i.e., speech and biological information.

### Third Embodiment

In the third embodiment, the information presentation apparatus (system) according to the first or second embodiment is applied to an entertainment apparatus (system) that presents moving image contents such as a game, movie, or the like. With this apparatus (system), development of the moving image contents is automatically controlled (changed) on the basis of the condition level of the physical/mental condition of the user (viewer) detected by the physical/mental condition detection unit 20. The arrangement and operation of the third embodiment will be explained below using the information presentation apparatus of the first embodiment.

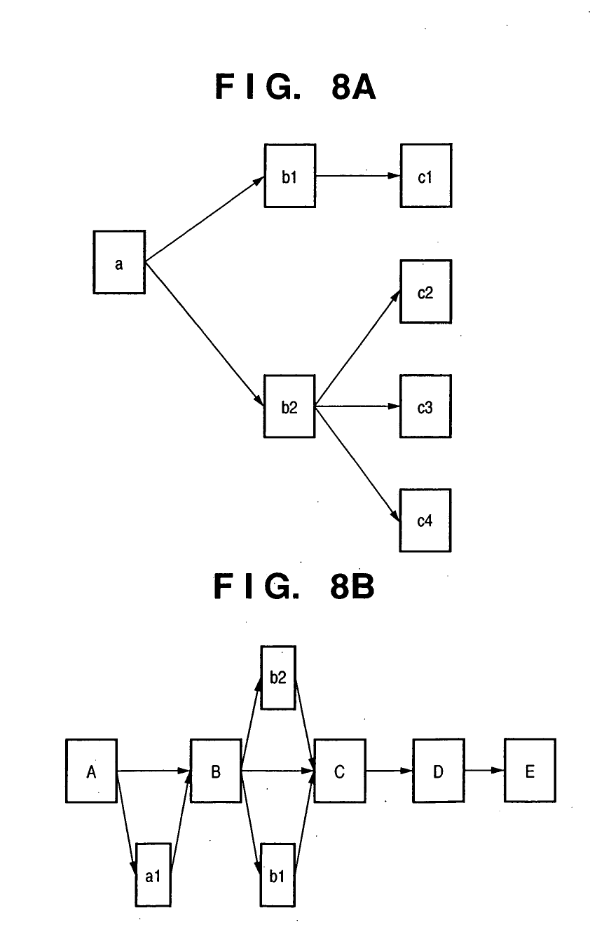

FIGS. 8A and 8B are views for explaining configuration examples of the moving image contents stored in the database unit 50. In the example of FIG. 8A, four different stories that start from a and finally arrive at last one of c1 to c4 are prepared. At the end of a as a part of these stories, the condition level of the physical/mental condition of the user is detected, and one of b1 and b2 is selected as the next story development. At the end of b2, one of stories c2 to c4 is similarly selected according to the condition level of the physical/mental condition. Alternatively, as shown in FIG. 8B, in story development from A to D, the condition level of the physical/mental condition is checked in a predetermined scene, and a story such as a1, b1, and the like may be added in accordance with the checking result.

That is, the condition level of the physical/mental condition of the user (viewer) is recognized in each of a plurality of scenes which are set in advance in the moving image contents, and the display content of the contents is controlled on the basis of the recognition result. As has been explained in the first embodiment, the physical/mental condition detection unit 20 detects the condition level on the basis of the detection result of a facial expression or action (nod, punching pose, crying, laughing) of the user by the gesture detection unit 303 and facial expression detection unit 302 included in the image recognition unit 15, or the conditions of biological signals (increases in heart rate, blood pressure, respiration frequency, sweating amount, and the like), and display development of the moving image is changed in accordance with this detection result. For example, in a scene in which a person in the moving image asks the user a question, the viewer's reaction (facial expression or gesture) is determined by the image recognition unit 15. If it is determined that the determination result corresponds to one of condition classes prepared in advance (affirmation/negation, satisfaction/dissatisfaction, interest/disinterest, happy/sad, and so forth), predetermined story development is made on the basis of the correspondence between the contents of that scene and the condition class of the physical/mental condition of the viewer. Also, when an abnormality of biological information (heart rate, blood pressure, or the like) is detected, a moving image development control program immediately aborts moving image display, displays an alert message, and so forth as in the first embodiment. Alternatively, the horror condition of the user is detected, and whether or not a predetermined horror scene is presented is determined by checking if that horror condition exceeds a given level. The story development control (i.e., information presentation control) may be made so that the biological feedback level falls within a predetermined range. For example, the upper and lower limit values are defined as an allowable range of the biological feedback level associated with an excitation level, fatigue level, or the like, a plurality of story developments are pre-set at each branch point in accordance with directionality indicating a direction to increase or decrease the excitation level or fatigue level, and the magnitude of the change, and the story development which has a direction to approach the median of the allowable range is selected.

### Fourth Embodiment

In the fourth embodiment, the information presentation apparatus (system) according to the first or second embodiment is applied to a robot. For example, the robot has arms, legs, a head, a body, and the like, the image sensing unit 10 and speech sensing unit 11 are provided to the head, and the biological information sensing unit 12 is provided to the hands. With this layout, the image of the user can be efficiently captured, and biological information can be acquired from the “hands” that can naturally contact the user. Note that pairs of right and left image sensing units and speech sensing units are provided. Since the pairs of right and left image and speech sensing units are provided to the head of the robot, perception of the depth distribution and three-dimensional information, estimation of the sound source direction, and the like can be achieved. The physical/mental condition detection unit 20 estimates the physical/mental condition of the nearby user on the basis of the obtained sensing information of the user, and information presentation is controlled in accordance with the estimation result.

### Fifth Embodiment

In the fifth embodiment, the information presentation system in the first embodiment is embedded in a display, wall/ceiling surface, window, mirror, or the like, and is hidden or discreet from the user. The display, wall/ceiling surface, window, mirror, or the like is made up of a translucent member, and allows to input an image of the user. Of the sensing units shown in FIG. 1, the image sensing unit (having a function as an input unit of a facial image and iris image) and speech sensing unit 11 are set on the information presentation system side. The biological information sensing unit 12 includes an expiratory sensor, blood pressure sensor, heart rate sensor, body temperature sensor, respiration pattern sensor, and the like, incorporates a communication unit as in the first embodiment, and is worn by the user (a living body such as a person, pet, or the like).

In this case, in particular, the physical/mental condition detection unit 20 estimates the health condition of the user on the basis of data such as the facial expression, gesture, expiration, iris pattern, blood pressure, and the like of the user. The information presentation unit 30 makes information presentation such as information presentation associated with the health condition of the user, advice presentation, and the like by means of text display on a display or an audible message from a loudspeaker. As for diagnosis of diseases based on exhalation, see the article of Nikkei Science, February 2004, p. 132 to 133 for reference. In addition, the control unit 40 has the same functions as in the first embodiment. The biological information sensing unit 12 includes a sensor unit which is worn by the user, and transmits an acquired signal, and a communication unit incorporated in the information presentation apparatus. A biological signal measured and acquired by the sensor unit is provided to the physical/mental condition detection unit 20 of the information presentation apparatus.

The aforementioned information presentation system may be used in apparatus environment settings in which the physical/mental condition detection unit which has an evaluation function of recognizing the facial expression of the user and evaluating a cheerful (or gloomy) expression is used, and the control unit controls to increase the brightness of a display or illumination as the recognized facial expression has a higher cheerful level, in accordance with the cheerfulness of the detected facial expression.

Note that the objects of the present invention are also achieved by supplying a storage medium, which records a program code of a software program that can implement the functions of the above-mentioned embodiments to the system or apparatus, and reading out and executing the program code stored in the storage medium by a computer (or a CPU or MPU) of the system or apparatus.

In this case, the program code itself read out from the storage medium implements the functions of the above-mentioned embodiments, and the storage medium which stores the program code constitutes the present invention.

As the storage medium for supplying the program code, for example, a flexible disk, hard disk, optical disk, magneto-optical disk, CD-ROM, CD-R, magnetic tape, nonvolatile memory card, ROM, and the like may be used.

The functions of the above-mentioned embodiments may be implemented not only by executing the readout program code by the computer but also by some or all of actual processing operations executed by an OS (operating system) running on the computer on the basis of an instruction of the program code.

Furthermore, the functions of the above-mentioned embodiments may be implemented by some or all of actual processing operations executed by a CPU or the like arranged in a function extension board or a function extension unit, which is inserted in or connected to the computer, after the program code read out from the storage medium is written in a memory of the extension board or unit.

According to the embodiments mentioned above, information associated with facial expressions and actions obtained from the image information can be used, and tacit physical/mental condition can be precisely detected. Also, according to the present invention, since speed and/or biological information can be used together with the information associated with facial expressions and actions in a comprehensive manner, and information presentation corresponding to the user's condition can be controlled by precisely detecting tacit physical/mental condition.

As many apparently widely different embodiments of the present invention can be made without departing from the spirit and scope thereof, it is to be understood that the invention is not limited to the specific embodiments thereof except as defined in the appended claims.

### CLAIM OF PRIORITY

This application claims priority from Japanese Patent Application No. 2004-049934 filed Feb. 25, 2004, which is hereby incorporated by reference herein.

1. An information processing apparatus comprising:
a first detection unit configured to detect a facial expression and/or body action of a user included in image information;
a determination unit configured to determine a physical/mental condition of a user on the basis of the detection result of said first detection unit;
a presentation unit configured to visually and/or audibly present information; and
a control unit configured to control presentation of the information by said presentation unit on the basis of the physical/mental condition of the user determined by said determination unit.

2. The apparatus according to claim 1, further comprising:
a second detection unit configured to detect speech and/or biological information of the user, and
wherein said determination unit determines the physical/mental condition of the user on the basis of detection results of said first and second detection units.

3. The apparatus according to claim 2, wherein said determination unit comprises:
a classification unit configured to classify the current physical/mental condition of the user to one of a plurality of classes defined in advance in association with physical/mental conditions of the user on the basis of information obtained by said first detection unit; and
a leveling unit configured to determine a level of the current physical/mental condition in the class classified by said classification unit on the basis of information obtained by said second detection unit.

4. The apparatus according to claim 2, wherein said determination unit comprises:
an extraction unit configured to extract as candidates some of a plurality of classes defined in advance in association with physical/mental conditions of the user on the basis of information obtained by said first detection unit; and
a decision unit configured to classify the current physical/mental condition of the user to one of the classes extracted by said extraction unit, and deciding a level of the physical/mental condition in the classified class.

5. The apparatus according to claim 2, further comprising:
a specifying unit configured to specify a user included in the image information; and
an acquisition unit configured to acquire individual information to be used in said determination unit on the basis of the specified user.

6. The apparatus according to claim 2, wherein the biological information includes at least some of a sweating level, pulsation, heart rate, respiration pattern, blood pressure, body temperature, pupil diameter, and iris pattern.

7. The apparatus according to claim 1, wherein when it is determined that the physical/mental condition determined by said determination unit corresponds to a condition defined as a dangerous condition, said control unit changes an information presentation content or aborts an information presentation operation.

8. The apparatus according to claim 1, wherein said presentation unit presents information detected in association with the physical/mental condition of the user.

9. The apparatus according to claim 1, wherein said presentation unit acquires information to be presented from an external apparatus.

10. The apparatus according to claim 1, further comprising:
a holding unit configured to, when the physical/mental condition of the user determined by said determination unit corresponds to a predetermined condition, holding an image of the user at that time, and
wherein said presentation unit presents a composite image generated by compositing an image acquired from an external apparatus to the image of the user held by said holding unit when it is determined that the physical/mental condition of the user corresponds to the predetermined condition.

11. The apparatus according to claim 1, wherein said control unit controls a presentation content by said presentation unit so that the physical/mental condition of the user determined by said determination uit falls within a predetermined level range.

12. The apparatus according to claim 1, wherein said presentation unit continuously presents a plurality of images or presents a moving image, and
said control unit controls to decide a presentation content on the basis of the physical/mental condition of the user determined by said determination unit.

13. An information processing method comprising:
a first detection step of detecting a facial expression and/or body action of a user included in image information;
a determination step of determining a physical/mental condition of a user on the basis of the detection result in the first detection step;
a presentation step of visually and/or audibly presenting information; and
a control step of controlling presentation of the information in the presentation step on the basis of the physical/mental condition of the user determined in the determination step.

14. The method according to claim 13, further comprising:
a second detection step of detecting speech and/or biological information of the user, and
wherein the determination step includes a step of determining the physical/mental condition of the user on the basis of detection results in the first and second detection steps.

15. A control program for making a computer execute an information processing method of claim 13.

16. A storage medium storing a control program for making a computer execute an information processing method of claim 13.
